(19) United States
(12) Patent Application Publication
(10) Pub. No.: US 2026/0256614 A1
(43) Pub. Date: Sep. 3, 2026

(54) MENSTRUAL RECEPTACLE HOLDERS, METHOD OF STORING MENSTRUAL RECEPTACLES, AND METHODS OF CLEANING MENSTRUAL RECEPTACLES

(71) Applicant: Cleva LLC, Des Moines, IA (US)

(72) Inventor: Christine Tran, Des Moines, IA (US)

(21) Appl. No.: 19/551,067

(22) Filed: Feb. 26, 2026

Related U.S. Application Data
(60) Provisional application No. 63/764,846, filed on Feb. 28, 2025.

(51) Int. Cl. A61F 5/455

(52) U.S. Cl. CPC A61F 5/4553

(57) ABSTRACT

The present disclosure provides a menstrual receptacle holder. The menstrual receptacle holder includes a structure including a protrusion that is configured to support a menstrual receptacle, wherein the protrusion is formed of a nonporous material. Also, a method of storing a menstrual receptacle holder is provided, including placing a menstrual receptacle over the protrusion of a menstrual receptacle holder. Further, a method of cleaning a menstrual receptacle is provided, including applying a cleaner to at least one of the protrusion of a menstrual receptacle holder or a menstrual receptacle and placing the menstrual receptacle on the protrusion.

### BACKGROUND

Sustainability has been a growing megatrend in the global economy. Consumers are increasingly discerning and sensitive to the environmental impact based on their purchasing choices. As a result, there has been a growing demand for sustainable products in the market. One such class of products that would benefit from incorporating reusable products is feminine hygiene products.

Menstrual receptacles, for instance menstrual cups and menstrual discs, which collect a volume of fluid during use in a non-absorbent vessel, provide certain advantages over absorbent menstrual products such as sanitary napkins and tampons. Such advantages include, for example, being more environmentally friendly and/or cost-effective due to typically being reusable, as well as decreasing at least one of odor, leakage, or risk of adverse health issues (e.g., dehydration, toxic shock syndrome, etc.). To increase the utilization of menstrual receptacles, there remains a need for greater ease and convenience in storing and cleaning menstrual receptacles.

### SUMMARY

In a first aspect, a menstrual receptacle holder is provided. The menstrual receptacle holder comprises a structure comprising a protrusion that is configured to support a menstrual receptacle, wherein the protrusion is formed of a nonporous material.

In a second aspect, a method of storing a menstrual receptacle holder is provided. The method comprises placing a menstrual receptacle over the protrusion of any menstrual receptacle holder according to any embodiment of the first aspect.

In a third aspect, a method of cleaning a menstrual receptacle. The method comprises applying a cleaner to at least one of the protrusion of any menstrual receptacle holder according to any embodiment of the first aspect or a menstrual receptacle; and placing the menstrual receptacle on the protrusion.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIGS. 1-24 are each generalized schematic perspective views of exemplary menstrual receptacle holders, according to various exemplary embodiments disclosed herein.

FIGS. 25A-25F are photographs of exemplary menstrual receptacle holders, according to various exemplary embodiments disclosed herein.

FIGS. 26A-26C are generalized schematic perspective views of exemplary covers for a menstrual receptacle holder.

FIG. 27 is a photograph of the menstrual receptacle holder of FIG. 25F with a menstrual cup placed on the protrusion of the menstrual receptacle holder.

FIG. 28 is a photograph of the menstrual receptacle holder of FIG. 25F with a menstrual disc placed on the protrusion of the menstrual receptacle holder.

In the drawings, like reference numerals indicate like elements. While the above-identified drawings, which may not be drawn to scale, set forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

For the following Glossary of defined terms, these definitions shall be applied for the entire application, unless a different definition is provided in the claims or elsewhere in the specification.

### Glossary

Certain terms are used throughout the description and the claims that, while for the most part are well known, may require some explanation. It should be understood that:

As used herein, “menstrual receptacle” refers to a menstrual product that is configured to collect and hold a volume of fluid within a continuous open area defined by at least one wall of the product. Menstrual cups and discs are common examples of menstrual receptacles. The continuous open area of menstrual receptacles is in contrast to absorbent menstrual products (e.g., pads, liners, tampons, etc.) that absorb and hold a volume of fluid in a discontinuous combination of absorbent materials (e.g., fibers, polymer particles, etc.) and interstitial space between the absorbent materials, as well as on exterior surface(s) of the absorbent product.

As used herein, “nonporous” means lacking voids (e.g., empty spaces and/or pores) and being impermeable to fluids.

As used herein, “fluid” refers to the physical states of matter of liquids and gases, and encompasses fluids including vapors, solvents, solutions, dispersions, and emulsions, including menstrual secretions.

As used herein, “support” with respect to support of a menstrual receptacle, means physically holding up the menstrual receptacle and/or providing a physical impediment to prevent the menstrual receptacle from sliding off the menstrual receptacle holder.

As used herein, “diameter” refers to a longest cross-sectional length that goes through a center of a protrusion. The cross-section of a protrusion in some cases will be circular and in other cases have a different shape.

The term “polymer” means a material that contains repeating chemical units, regularly or irregularly arranged. “polymeric” and “plastic” each mean a material that mainly includes one or more polymers and may contain other components.

The term “at least one” includes all integers of one and greater (e.g., at least 2, at least 4, at least 6, at least 8, at least 10, at least 25, at least 50, at least 100, etc.).

The terms “about” or “approximately” with reference to a numerical value or a shape means +/− five percent of the numerical value or property or characteristic, but expressly includes the exact numerical value.

As used in this specification and the appended embodiments, the singular forms “a”, “an”, and “the” include plural referents unless the content clearly dictates otherwise. As used in this specification and the appended embodiments, the term “or” is generally employed in its sense including “and/or” unless the content clearly dictates otherwise.

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Various exemplary embodiments of the disclosure will now be described. Exemplary embodiments of the present disclosure may take on various modifications and alterations without departing from the spirit and scope of the present disclosure. Accordingly, it is to be understood that the embodiments of the present disclosure are not to be limited to the following described exemplary embodiments but is to be controlled by the limitations set forth in the claims and any equivalents thereof.

As mentioned above, menstrual receptacles have numerous advantages over other menstrual products, particularly over single-use absorbent products. However, most menstrual receptacles come with, e.g., a cloth bag at purchase but lack a hygienic holder for storage in between uses. Additionally, reusable menstrual receptacles are often boiled or steamed during a cleaning process and need to be placed somewhere clean and temperature-resistant to cool. It has been discovered that it is possible to provide a hygienic nonporous menstrual receptacle holder to solve at least some of the above-identified issues.

Exemplary suitable nonporous materials include both materials that themselves are nonporous and porous materials having an impermeable coating thereon (e.g., on a major surface of the porous material). For instance, silicone, stainless steel, aluminum, and glass are each a nonporous material. In addition to silicone, other nonporous polymeric materials (e.g., plastics) include for instance and without limitation, polyethylene (PE) (e.g., high-density polyethylene), polypropylene (PP), polystyrene (PS), polyvinyl chloride (PVC), acrylonitrile butadiene styrene (ABS), polycarbonate (PC), polyethylene terephthalate (PET), polymethyl methacrylate (PMMA), polyamide (PA), and polyoxymethylene (POM). Bamboo may also be a suitable material due to being dense and moisture resistant.

Exemplary suitable nonporous materials that are porous materials having an impermeable coating thereon include for instance and without limitation, glazed ceramics. Some exemplary ceramic materials include for instance and without limitation, the clays of kaolin, ball clay, earthenware, stoneware, and porcelain. Various additives may also be included, such as silica and/or feldspar. Exemplary suitable glazes include for instance and without limitation, glossy glazes, matte glazes, satin glazes, transparent glazes, opaque glazes, celadon glazes, tenmoku glazes, and shino glazes. Some typical components of a glaze include silica, alumina, one or more fluxes (e.g., feldspar, lead oxide, and/or a borate), optionally a colorant, optionally an opacifier (e.g., tin oxide and/or zirconium oxide), and optionally one or more stabilizers (e.g., calcium oxide and/or magnesium oxide). In some embodiments, it may be preferable to use a glaze that is food safe.

Other suitable nonporous coatings may be used with a porous material, or even a nonporous material that would benefit from a hard polymeric coating (e.g., a lacquer), include for instance and without limitation, acrylic coatings, polyurethane coatings, and epoxy coatings.

The menstrual receptacle holder may be manufactured via injection molding, blow molding, metal casting, slip casting from a mold, additive manufacturing (e.g., of clay or plastic), ceramic wheel throwing, and ceramic hand building, as known to those of skill in the art. When a polymeric coating is subsequently applied, it may be applied by known methods such as brushing, spraying, dipping, pouring, rolling, spreading, flow coating, etc. Glazing processes for ceramic materials are also well known in the art.

### Menstrual Receptacle Holders

In a first aspect, a menstrual receptacle holder is provided. The menstrual receptacle holder comprises a structure comprising a protrusion that is configured to support a menstrual receptacle, wherein the protrusion is formed of a nonporous material. As menstrual receptacles typically have a curvilinear cross-sectional shape, in many embodiments the protrusion of a menstrual receptacle holder has a curvilinear cross-sectional shape.

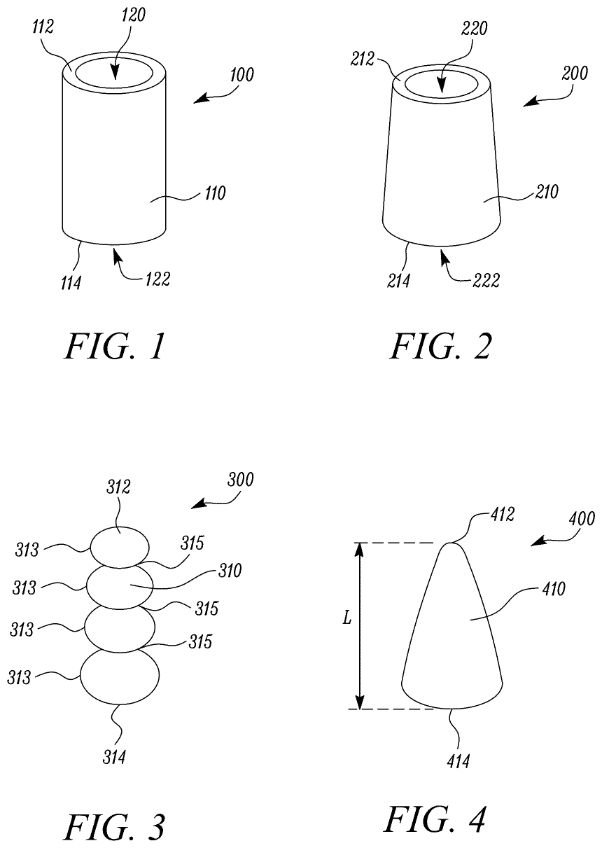

Referring to FIG. 1, a generalized schematic perspective view of an exemplary menstrual receptacle holder 100 is provided. The menstrual receptacle holder 100 includes a structure comprising a protrusion 110 that is configured to support a menstrual receptacle. The menstrual receptacle holder 100 of this embodiment has a protrusion 110 defining a hollow body defining an open first end 120 and an open opposing second end 122. A top end 112 and a bottom end 114 of the protrusion 110 are also indicated. This protrusion 110 has a cylindrical shape. Further, the structure consists of the protrusion 110.

Referring to FIG. 2, a generalized schematic perspective view of an exemplary menstrual receptacle holder 200 is provided. The menstrual receptacle holder 200 includes a structure comprising a protrusion 210 that is configured to support a menstrual receptacle. The menstrual receptacle holder 200 of this embodiment has a protrusion 210 defining a hollow body defining an open first end 220 and an open opposing second end 222. A top end 212 and a bottom end 214 of the protrusion 210 are also indicated. This protrusion 210 has a frustoconical shape. Further, the structure consists of the protrusion 210.

Referring to FIG. 3, a generalized schematic perspective view of an exemplary menstrual receptacle holder 300 is provided. The menstrual receptacle holder 300 includes a structure comprising a protrusion 310 that is configured to support a menstrual receptacle. The protrusion 310 of this embodiment is solid. Further, the structure consists of the protrusion 310. Additionally, the protrusion 310 of this embodiment has a plurality of horizontal utilitarian protrusions 313 and indentations 315. This is in contrast to a plurality of vertical utilitarian protrusions and indentations such as those found on a fruit juicer or reamer and make the device operational to extract juice from fruit. Typically, the menstrual receptacle holders according to the present disclosure have a protrusion with a main body that is free of vertical utilitarian protrusions and indentations.

Referring to FIG. 4, a generalized schematic perspective view of an exemplary menstrual receptacle holder 400 is provided. The menstrual receptacle holder 400 includes a structure comprising a protrusion 410 that is configured to support a menstrual receptacle. The protrusion 410 of this embodiment is solid and has a conical shape. Further, the structure consists of the protrusion 410.

As depicted in FIG. 4, in some embodiments, a menstrual receptacle holder that consists of the protrusion 410 includes a protrusion 410 having a length L from a top end 412 to an opposing bottom end 414 of 40 millimeters (mm) or greater, such as 45 mm, 50 mm, 55 mm, 60 mm, or 70 mm or greater; and 100 mm or less, 95 mm, 90 mm, 85 mm, 80 mm, 75 mm, 70 mm, 65 mm, 60 mm, 55 mm, or 50 mm or less. In some cases, the protrusion has a length L from a top end to an opposing bottom end of 40 mm to 100 mm. Having a height within this range may advantageously prevent a menstrual receptacle that is being supported by the protrusion from coming into contact with a non-sterile surface on which the menstrual receptacle holder is placed. For menstrual receptacles such as menstrual discs, a shorter protrusion may be used, whereas for menstrual receptacles such as menstrual cups, a longer protrusion may be used.

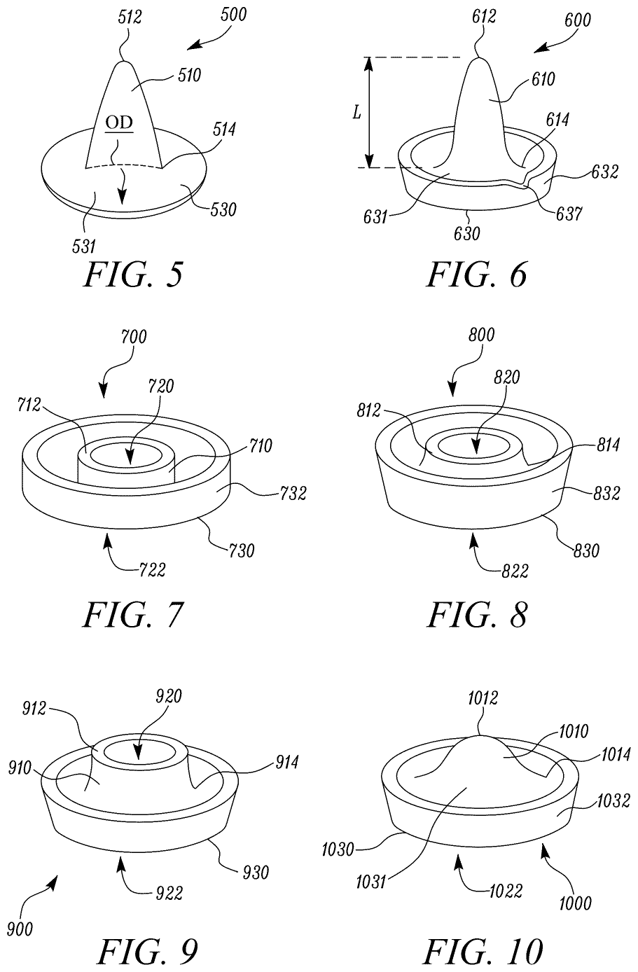

Referring to FIG. 5, a generalized schematic perspective view of an exemplary menstrual receptacle holder 500 is provided. The menstrual receptacle holder 500 includes a structure comprising a protrusion 510 that is configured to support a menstrual receptacle. The menstrual receptacle holder 500 of this embodiment has a protrusion 510 comprising a first end 512 and an opposing second end 514 and the structure further comprises a base 530 attached to the second end 514 of the protrusion 510. The base 530 comprises a portion 531 that extends in a direction away from a center point of the protrusion 510. In this embodiment, the base 530 is flat. The inclusion of a base may be advantageous in preventing a menstrual receptacle from coming into contact with a dirty or non-sterile surface.

As depicted in FIG. 5, in some embodiments, a menstrual receptacle holder includes a protrusion 510 having a largest outer diameter OD of 10 mm or greater, such as 15 mm, 20 mm, 25 mm, 30 mm, 35 mm, 40 mm, 45 mm, or 50 mm or greater; and 70 mm or less, 65 mm, 60 mm, 55 mm, 50 mm, 45 mm, or 40 mm or less. In some cases, the protrusion has a largest outer diameter OD of 10 mm to 70 mm.

Referring to FIG. 6, a generalized schematic perspective view of an exemplary menstrual receptacle holder 600 is provided. The menstrual receptacle holder 600 includes a structure comprising a protrusion 610 that is configured to support a menstrual receptacle. The menstrual receptacle holder 600 of this embodiment has a protrusion 610 comprising a first end 612 and an opposing second end 614 and the structure further comprises a base 630 attached to the second end 614 of the protrusion 610. The base 630 comprises a portion 631 that extends in a direction away from a center point of the protrusion 610. Additionally, the base 630 further comprises a wall 632 that extends in a direction away from the portion 631 and provides an exterior structure of the menstrual receptacle holder. The direction can vary, such as extending away from the portion at an angle from 5 degrees to 120 degrees. Further, the height of the wall 632 may vary, such as from 2 mm to 150 mm. Providing a wall on a base may advantageously minimize transfer of any fluid (e.g., water) from the holder to a surface on which the holder is placed following cleaning of the menstrual receptacle, when the menstrual receptacle is still wet. In the embodiment of FIG. 6, the wall 632 includes an optional spout 637 to facilitate pouring fluid out of the base 630.

As depicted in FIG. 6, in some embodiments, a menstrual receptacle holder that includes both a protrusion 610 and a base 630 includes a protrusion 610 having a length L from a top end 612 to an opposing bottom end 614 of 20 mm or greater, such as 25 mm, 30 mm, 35 mm, 40 mm, 45 mm, 50 mm, 55 mm, 60 mm, or 70 mm or greater; and 100 mm or less, 95 mm, 90 mm, 85 mm, 80 mm, 75 mm, 70 mm, 65 mm, 60 mm, 55 mm, or 50 mm or less. In some cases, the protrusion has a length L from a top end to an opposing bottom end of 20 mm to 100 mm.

Referring to FIG. 7, a generalized schematic perspective view of an exemplary menstrual receptacle holder 700 is provided. The menstrual receptacle holder 700 includes a structure comprising a protrusion 710 that is configured to support a menstrual receptacle. The menstrual receptacle holder 700 of this embodiment has a protrusion 710 defining a hollow body defining an open first end 720 and an open opposing second end 722. This protrusion 710 has a cylindrical shape. The menstrual receptacle holder 700 of this embodiment has a protrusion 710 comprising a first end 712 and an opposing second end (not shown) and the structure further comprises a base 730 attached to the second end of the protrusion 710. The base 730 comprises a portion (not shown) that extends in a direction away from a center point of the protrusion 710. Additionally, the base 730 further comprises a wall 732 that extends in a direction away from the portion and provides an exterior structure of the menstrual receptacle holder 700. The wall 732 has a single height and the height is shorter than the length of the protrusion 710.

Referring to FIG. 8, a generalized schematic perspective view of an exemplary menstrual receptacle holder 800 is provided. The menstrual receptacle holder 800 includes a structure comprising a protrusion 810 that is configured to support a menstrual receptacle. The menstrual receptacle holder 800 of this embodiment has a protrusion 810 defining a hollow body defining an open first end 820 and an open opposing second end 822. This protrusion 810 has a frustoconical shape. The menstrual receptacle holder 800 of this embodiment has a protrusion 810 comprising a first end 812 and an opposing second end 814 and the structure further comprises a base 830 attached to the second end 814 of the protrusion 810. The base 830 comprises a portion (not shown) that extends in a direction away from a center point of the protrusion 810. Additionally, the base 830 further comprises a wall 832 that extends in a direction away from the portion and provides an exterior structure of the menstrual receptacle holder 800. The wall 832 has a single height that is equal to the length of the protrusion 810.

Referring to FIG. 9, a generalized schematic perspective view of an exemplary menstrual receptacle holder 900 is provided. The menstrual receptacle holder 900 includes a structure comprising a protrusion 910 that is configured to support a menstrual receptacle. The menstrual receptacle holder 900 of this embodiment has a protrusion 910 defining a hollow body defining an open first end 920 and an open opposing second end 922. This protrusion 910 has a frustoconical shape. The menstrual receptacle holder 900 of this embodiment has a protrusion 910 comprising a first end 912 and an opposing second end 914 and the structure further comprises a base 930 attached to the second end 914 of the protrusion 910. The base 930 comprises a portion (not shown) that extends in a direction away from a center point of the protrusion 910. Additionally, the base 930 further comprises a wall 932 that extends in a direction away from the portion and provides an exterior structure of the menstrual receptacle holder 900. The wall 932 has a single height and the height is shorter than the length of the protrusion 910.

Referring to FIG. 10, a generalized schematic perspective view of an exemplary menstrual receptacle holder 1000 is provided. The menstrual receptacle holder 1000 includes a structure comprising a protrusion 1010 that is configured to support a menstrual receptacle. The menstrual receptacle holder 1000 of this embodiment has a protrusion 1010 comprising a first end 1012 and an opposing second end 1014 and the structure further comprises a base 1030 attached to the second end 1014 of the protrusion 1010. The base 1030 comprises a portion 1031 that extends in a direction away from a center point of the protrusion 1010. Additionally, the base 1030 further comprises a wall 1032 that extends in a direction away from the portion and provides an exterior structure of the menstrual receptacle holder 1000. The wall 1032 has a single height and the height is shorter than the length of the protrusion 1010. Further, the protrusion 1010 has a hollow interior such that there is an open end 1022 opposite the first end 1012 of the protrusion 1010.

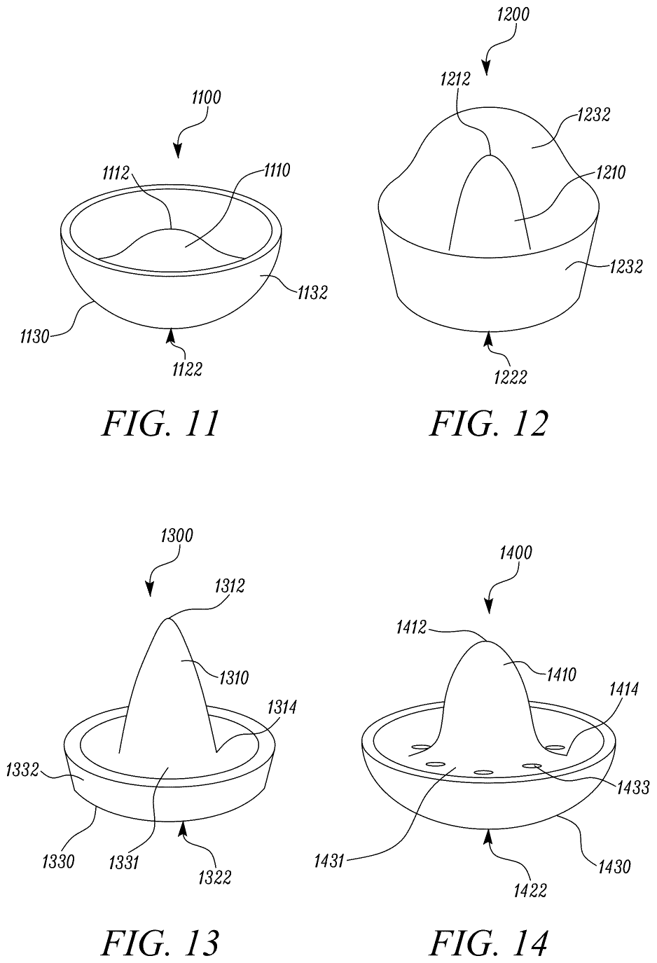

Referring to FIG. 11, a generalized schematic perspective view of an exemplary menstrual receptacle holder 1100 is provided. The menstrual receptacle holder 1100 includes a structure comprising a protrusion 1110 that is configured to support a menstrual receptacle. The menstrual receptacle holder 1100 of this embodiment has a protrusion 1110 comprising a first end 1112 and an opposing second end 1114 and the structure further comprises a base 1130 attached to the second end 1114 of the protrusion 1110. The base 1130 comprises a portion (not shown) that extends in a direction away from a center point of the protrusion 1110. Additionally, the base 1130 further comprises a wall 1132 that extends in a direction away from the portion and provides an exterior structure of the menstrual receptacle holder 1100. The wall 1132 is curved, has a single height, and the height is taller than the length of the protrusion 1110. Further, the protrusion 1110 has a hollow interior such that there is an open end 1122 opposite the first end 1112 of the protrusion 1110.

Referring to FIG. 12, a generalized schematic perspective view of an exemplary menstrual receptacle holder 1200 is provided. The menstrual receptacle holder 1200 includes a structure comprising a protrusion 1210 that is configured to support a menstrual receptacle. The menstrual receptacle holder 1200 of this embodiment has a protrusion 1210 comprising a first end 1212 and an opposing second end (not shown) and the structure further comprises a base 1230 attached to the second end of the protrusion 1210. The base 1230 comprises a portion (not shown) that extends in a direction away from a center point of the protrusion 1210. Further, the protrusion 1210 has a hollow interior such that there is an open end 1222 opposite the first end 1212 of the protrusion 1210. Additionally, the base 1230 further comprises a wall 1232 that extends in a direction away from the portion and provides an exterior structure of the menstrual receptacle holder 1200. The wall 1232 has more than two heights, at least one of which is shorter than the length of the protrusion 1210 and at least one of which is taller than the length of the protrusion 1210. The inclusion of one or more tall sections of wall may be desirable to shield the menstrual receptacle from view when placed on the menstrual receptacle holder (and e.g., positioned appropriately). The inclusion of one or more short sections of wall may be desirable to make it easier to wash the menstrual receptacle as compared to a tall wall having a single height.

Referring to FIG. 13, a generalized schematic perspective view of an exemplary menstrual receptacle holder 1300 is provided. The menstrual receptacle holder 1300 includes a structure comprising a protrusion 1310 that is configured to support a menstrual receptacle. The menstrual receptacle holder 1300 of this embodiment has a protrusion 1310 comprising a first end 1312 and an opposing second end 1314 and the structure further comprises a base 1330 attached to the second end 1314 of the protrusion 1310. The base 1330 comprises a portion 1331 that extends in a direction away from a center point of the protrusion 1310. Additionally, the base 1330 further comprises a wall 1332 that extends in a direction away from the portion and provides an exterior structure of the menstrual receptacle holder 1300. The wall 1332 has a single height and the height is shorter than the length of the protrusion 1310. Further, the protrusion 1310 has a hollow interior such that there is an open end 1322 opposite the first end 1312 of the protrusion 1310.

Referring to FIG. 14, a generalized schematic perspective view of an exemplary menstrual receptacle holder 1400 is provided. The menstrual receptacle holder 1400 includes a structure comprising a protrusion 1410 that is configured to support a menstrual receptacle. The menstrual receptacle holder 1400 of this embodiment has a protrusion 1410 comprising a first end 1412 and an opposing second end 1414 and the structure further comprises a base 1430 attached to the second end 1414 of the protrusion 1410. The base 1430 comprises a portion 1431 that extends in a direction away from a center point of the protrusion 1410. Additionally, the base 1430 further comprises a wall 1432 that extends in a direction away from the portion and provides an exterior structure of the menstrual receptacle holder 1400. The wall 1432 is curved, has a single height, and the height is shorter than the length of the protrusion 1410. Further, the protrusion 1410 has a hollow interior such that there is an open end 1422 opposite the first end 1412 of the protrusion 1410. As depicted in FIG. 14, in some embodiments, the portion 1431 of the base 1430 defines at least one void 1433 through a thickness of the portion 1431. Including at least one void (e.g., hole or aperture) allows for fluid (e.g., water) to drain away from the protrusion 1410 when a menstrual receptacle is placed on the protrusion 1410 while still wet from cleaning.

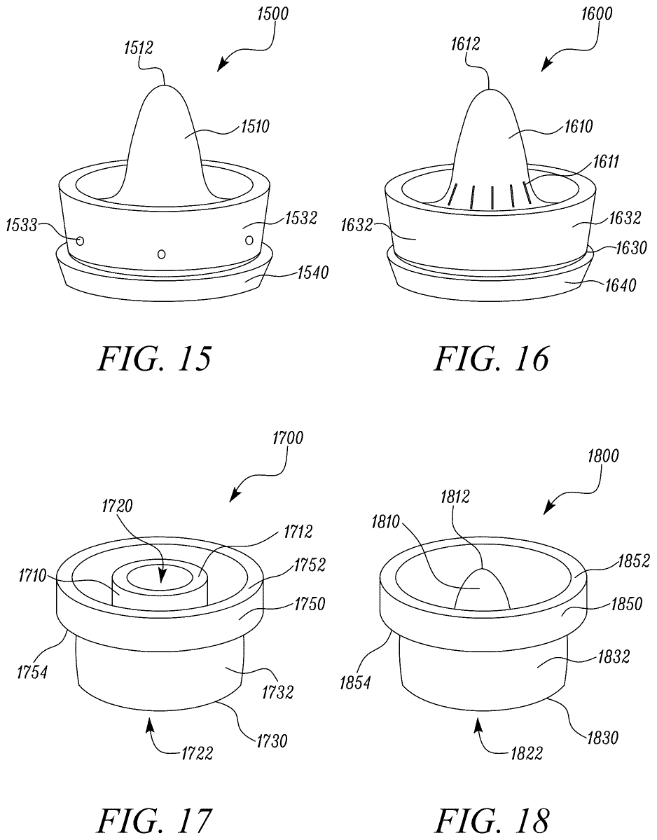

Referring to FIG. 15, a generalized schematic perspective view of an exemplary menstrual receptacle holder 1500 is provided. The menstrual receptacle holder 1500 includes a structure comprising a protrusion 1510 that is configured to support a menstrual receptacle. The menstrual receptacle holder 1500 of this embodiment has a protrusion 1510 comprising a first end 1512 and an opposing second end (not shown) and the structure further comprises a base 1530 attached to the second end of the protrusion 1510. The base 1530 comprises a portion (not shown) that extends in a direction away from a center point of the protrusion 1510. Additionally, the base 1530 further comprises a wall 1532 that extends in a direction away from the portion and provides an exterior structure of the menstrual receptacle holder 1500. The wall 1532 has a single height and the height is shorter than the length of the protrusion 1510. As depicted in FIG. 15, in some embodiments, the wall 1532 defines at least one void 1533 through a thickness of the wall 1532. The embodiment shown in FIG. 15 further includes a tray 1540 disposed beneath the base 1530. A tray may be a separate part, detachable from the base, or permanently attached to the base of a menstrual receptacle holder.

Referring to FIG. 16, a generalized schematic perspective view of an exemplary menstrual receptacle holder 1600 is provided. The menstrual receptacle holder 1600 includes a structure comprising a protrusion 1610 that is configured to support a menstrual receptacle. The menstrual receptacle holder 1600 of this embodiment has a protrusion 1610 comprising a first end 1612 and an opposing second end (not shown) and the structure further comprises a base 1630 attached to the second end of the protrusion 1610. The base 1630 comprises a portion (not shown) that extends in a direction away from a center point of the protrusion 1610. Additionally, the base 1630 further comprises a wall 1632 that extends in a direction away from the portion and provides an exterior structure of the menstrual receptacle holder 1600. The wall 1632 has a single height and the height is shorter than the length of the protrusion 1610. As depicted in FIG. 16, in some embodiments, the protrusion 1610 defines at least one void 1611 through a thickness of the protrusion 1610. The embodiment shown in FIG. 16 further includes a tray 1640 disposed beneath the base 1630, and the tray 1640 is attached to the base 1630.

Referring to FIG. 17, a generalized schematic perspective view of an exemplary menstrual receptacle holder 1700 is provided. The menstrual receptacle holder 1700 includes a structure comprising a protrusion 1710 that is configured to support a menstrual receptacle. The menstrual receptacle holder 1700 of this embodiment has a protrusion 1710 defining a hollow body defining an open first end 1720 and an open opposing second end 1722. This protrusion 1710 has a cylindrical shape. The menstrual receptacle holder 1700 of this embodiment has a protrusion 1710 comprising a first end 1712 and an opposing second end 1714 and the structure further comprises a base 1730 attached to the second end (not shown) of the protrusion 1710. The base 1730 comprises a portion (not shown) that extends in a direction away from a center point of the protrusion 1710. Additionally, the base 1730 further comprises a wall 1732 that extends in a direction away from the portion and provides an exterior structure of the menstrual receptacle holder 1700. The wall 1732 has a single height and the height is shorter than the length of the protrusion 1710. The wall 1732 also includes a lip 1750 that may advantageously assist in making the menstrual receptacle holder 1700 readily stackable with more menstrual receptacle holders having the same design, e.g., for shipping, storage, and/or display in a retail location. For instance, the wall 1732 of one menstrual receptacle holder 1700 may fit in between the protrusion 1710 and the lip 1750 of another menstrual receptacle holder and optionally a lower surface 1754 of the lip 1750 may rest on an upper surface (1752) of the menstrual receptacle holder on which the menstrual receptacle holder 1700 is stacked.

Referring to FIG. 18, a generalized schematic perspective view of an exemplary menstrual receptacle holder 1800 is provided. The menstrual receptacle holder 1800 includes a structure comprising a protrusion 1810 that is configured to support a menstrual receptacle. The menstrual receptacle holder 1800 of this embodiment has a protrusion 1810 comprising a first end 1812 and an opposing second end (not shown) and the structure further comprises a base 1830 attached to the second end of the protrusion 1810. The base 1830 comprises a portion (not shown) that extends in a direction away from a center point of the protrusion 1810. Additionally, the base 1830 further comprises a wall 1832 that extends in a direction away from the portion and provides an exterior structure of the menstrual receptacle holder 1800. The wall 1832 has a single height and the height is equal to the length of the protrusion 1810. The wall 1832 also includes a lip 1850 for stacking the holder with more menstrual receptacle holders having the same design. The lip 1850 has an upper surface 1852 and a lower surface 1854. Further, the protrusion 1810 has a hollow interior such that there is an open end 1822 opposite the first end 1812 of the protrusion 1810.

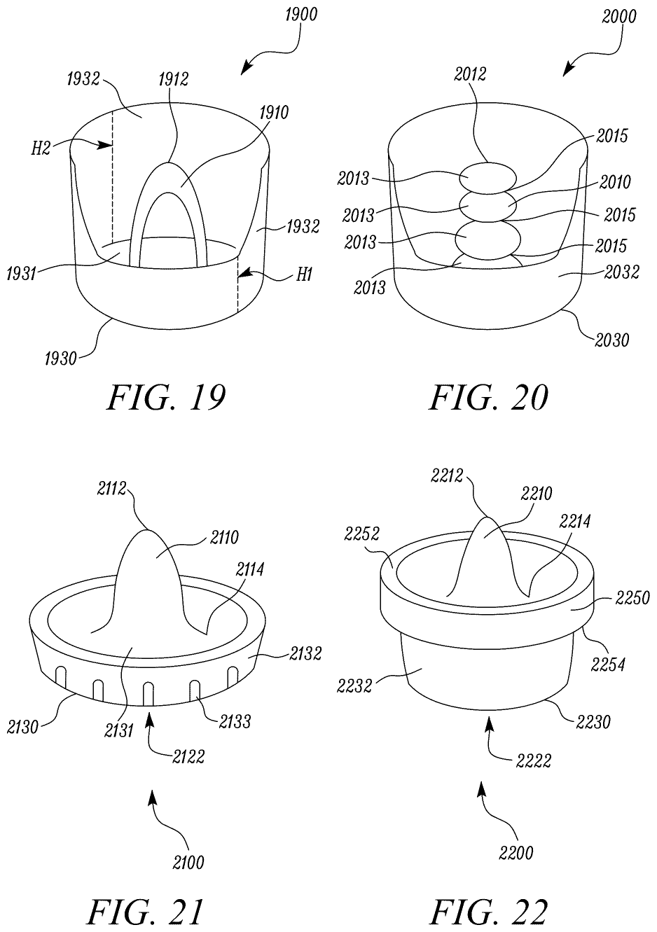

Referring to FIG. 19, a generalized schematic perspective view of an exemplary menstrual receptacle holder 1900 is provided. The menstrual receptacle holder 1900 includes a structure comprising a protrusion 1910 that is configured to support a menstrual receptacle. The menstrual receptacle holder 1900 of this embodiment has a protrusion 1910 comprising a first end 1912 and an opposing second end (not shown) and the structure further comprises a base 1930 attached to two second ends (not shown) of the protrusion 1910. The base 1930 comprises a portion 1931 that extends in a direction away from a center point of the protrusion 1910. Additionally, the base 1930 further comprises a wall 1932 that extends in a direction away from the portion and provides an exterior structure of the menstrual receptacle holder 1900. The wall 1932 has more than two heights, at least one of which, H₁, is shorter than the length of the protrusion 1910 and at least one of which, H₂, is taller than the length of the protrusion 1910.

Referring to FIG. 20, a generalized schematic perspective view of an exemplary menstrual receptacle holder 2000 is provided. The menstrual receptacle holder 2000 includes a structure comprising a protrusion 2010 that is configured to support a menstrual receptacle. The menstrual receptacle holder 2000 of this embodiment has a protrusion 2010 comprising a first end 2012 and an opposing second end (not shown). The protrusion 2010 of this embodiment is solid and has a plurality of horizontal utilitarian protrusions 2013 and indentations 2015. The structure further comprises a base 2030 attached to the second end of the protrusion 2010. The base 2030 comprises a portion 2031 that extends in a direction away from a center point of the protrusion 2010. Additionally, the base 2030 further comprises a wall 2032 that extends in a direction away from the portion and provides an exterior structure of the menstrual receptacle holder 2000. The wall 2032 has more than two heights, at least one of which is shorter the length of the protrusion 2010 and at least one of which is taller than the length of the protrusion 2010.

Referring to FIG. 21, a generalized schematic perspective view of an exemplary menstrual receptacle holder 2100 is provided. The menstrual receptacle holder 2100 includes a structure comprising a protrusion 2110 that is configured to support a menstrual receptacle. The menstrual receptacle holder 2100 of this embodiment has a protrusion 2110 comprising a first end 2112 and an opposing second end 2114 and the structure further comprises a base 2130 attached to the second end of the protrusion 2110. The base 2130 comprises a portion 2131 that extends in a direction away from a center point of the protrusion 2110. Additionally, the base 2130 further comprises a wall 2132 that extends in a direction away from the portion and provides an exterior structure of the menstrual receptacle holder 2100. The wall 2132 has a single height and the height is shorter than the length of the protrusion 2110. Further, the protrusion 2110 has a hollow interior such that there is an open end 2122 opposite the first end 2112 of the protrusion 2110. As depicted in FIG. 21, in some embodiments, the wall 2132 defines at least one void 2133 through a thickness of the wall 2132.

Referring to FIG. 22, a generalized schematic perspective view of an exemplary menstrual receptacle holder 2200 is provided. The menstrual receptacle holder 2200 includes a structure comprising a protrusion 2210 that is configured to support a menstrual receptacle. The menstrual receptacle holder 2200 of this embodiment has a protrusion 2210 comprising a first end 2212 and an opposing second end 2214 and the structure further comprises a base 2230 attached to the second end 2214 of the protrusion 2210. The base 2230 comprises a portion (not shown) that extends in a direction away from a center point of the protrusion 2210. Additionally, the base 2230 further comprises a wall 2232 that extends in a direction away from the portion and provides an exterior structure of the menstrual receptacle holder 2200. The wall 2232 has a single height and the height is shorter than the length of the protrusion 2210. The wall 2232 also includes a lip 2250 for stacking the holder with more menstrual receptacle holders having the same design. The lip 2250 has an upper surface 2252 and a lower surface 2254. Further, the protrusion 2210 has a hollow interior such that there is an open end 2222 opposite the first end 2212 of the protrusion 2210.

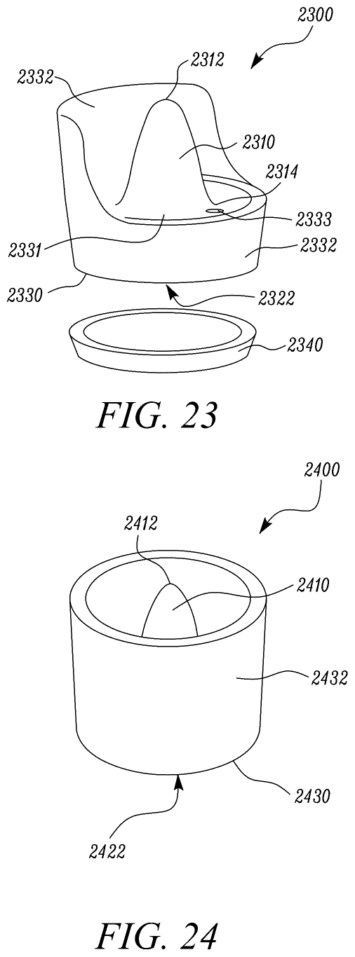

Referring to FIG. 23, a generalized schematic perspective view of an exemplary menstrual receptacle holder 2300 is provided. The menstrual receptacle holder 2300 includes a structure comprising a protrusion 2310 that is configured to support a menstrual receptacle. The menstrual receptacle holder 2300 of this embodiment has a protrusion 2310 comprising a first end 2312 and an opposing second end 2314 and the structure further comprises a base 2330 attached to the second end of the protrusion 2310. The base 2330 comprises a portion (not shown) that extends in a direction away from a center point of the protrusion 2310. Additionally, the base 2330 further comprises a wall 2332 that extends in a direction away from the portion and provides an exterior structure of the menstrual receptacle holder 2300. The wall 2332 has more than two heights, at least one of which is shorter than the length of the protrusion 2310 and at least one of which is taller than the length of the protrusion 2310. Further, the protrusion 2310 has a hollow interior such that there is an open end 2222 opposite the first end 2312 of the protrusion 2310. As depicted in FIG. 23, in some embodiments, the portion 2331 of the base 2330 defines at least one void 2333 through a thickness of the portion 2331. The embodiment shown in FIG. 23 further includes a separate tray 2340 on which the base 2330 may be placed.

Referring to FIG. 24, a generalized schematic perspective view of an exemplary menstrual receptacle holder 2400 is provided. The menstrual receptacle holder 2400 includes a structure comprising a protrusion 2410 that is configured to support a menstrual receptacle. The menstrual receptacle holder 2400 of this embodiment has a protrusion 2410 comprising a first end 2412 and an opposing second end (not shown) and the structure further comprises a base 2430 attached to the second end of the protrusion 2410. The base 2430 comprises a portion (not shown) that extends in a direction away from a center point of the protrusion 2410. Additionally, the base 2430 further comprises a wall 2432 that extends in a direction away from the portion and provides an exterior structure of the menstrual receptacle holder 2400. The wall 2432 has a single height and the height is equal to the length of the protrusion 2410. Further, the protrusion 2410 has a hollow interior such that there is an open end 2422 opposite the first end 2412 of the protrusion 2410.

In many embodiments, an interior of the protrusion is configured to provide a finger grip for the menstrual receptacle holder, such as provided by the designs depicted in each of FIGS. 5, 6, 12-16, 18, 21-24, and 25. For instance, referring to FIG. 6, there is an open end 622 at the base and the protrusion 610 has a hollow interior and has a shape that would allow the menstrual receptacle holder 600 to be readily gripped by a finger of a user. This may be advantageous when handling a clean (e.g., just washed) menstrual receptacle holder and/or when using the menstrual receptacle holder in a process of cleaning a menstrual receptacle.

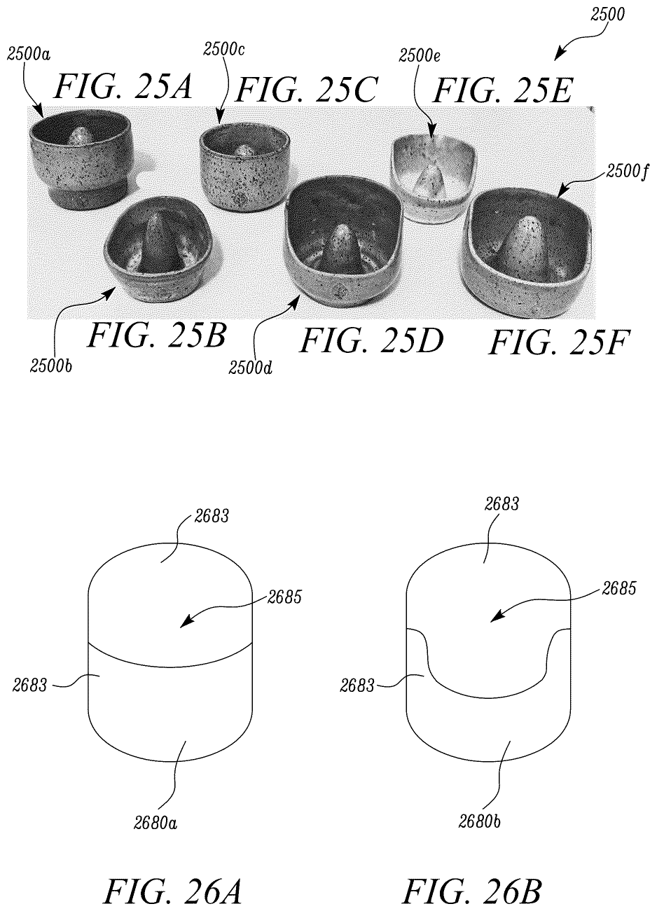

Referring to FIGS. 25A-25F, photographs are provided of exemplary menstrual receptacle holders 2500a, 2500b, 2500c, 2500d, 2500e, and 2500f, formed of glazed ceramic.

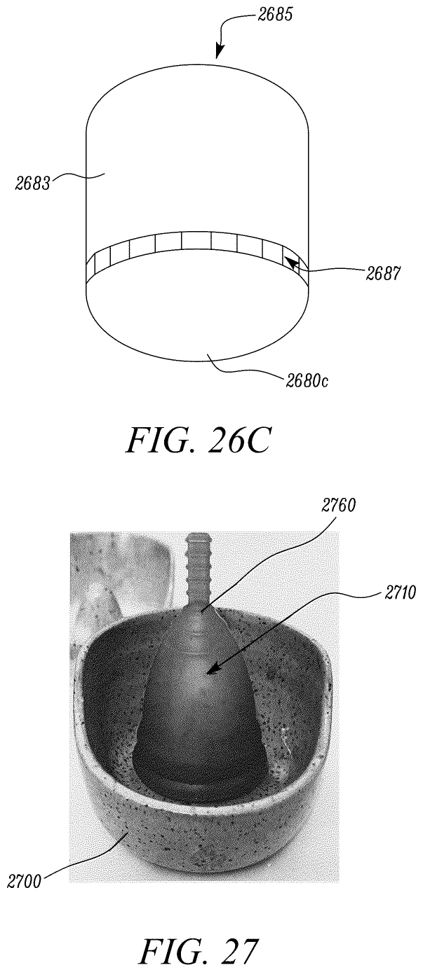

Referring to FIGS. 26A-26C, in some embodiments, a menstrual receptacle holder further comprises a cover configured to surround the protrusion, or in the case of the holder having a wall that is taller than the protrusion, configured to be placed on the wall. A cover can be useful to keep dust and debris from getting in the holder and/or to shield a menstrual receptacle from view when placed on the menstrual receptacle holder. FIG. 26A depicts one exemplary cover 2680a, configured to fit on a menstrual receptacle holder that has a wall with two different heights (e.g., the holder 2500f in FIG. 25F). The cover 2680a includes a wall 2683 with two different heights and defines an opening 2685 into which a menstrual receptacle holder protrusion may fit. Similarly, FIG. 26B depicts an exemplary cover 2680b, configured to fit on a menstrual receptacle holder that has a wall with more than two different heights. The cover 2680b includes a wall 2683 with numerous different heights and defines an opening 2685 into which a menstrual receptacle holder protrusion may fit. FIG. 26C depicts an exemplary cover 2680c configured to fit on a menstrual receptacle holder that has a wall with a single height (e.g., the holder 2500c in FIG. 25C). The cover 2680c includes a wall 2683 with the same height around the cover and defines an opening 2685 into which a menstrual receptacle holder protrusion may fit. Additionally, the cover 2680c includes a plurality of voids 2687 to assist in allowing moisture to exit the covered holder (e.g., for air drying of a wet menstrual receptacle located on a covered menstrual receptacle holder). It is noted that each of the covers of FIGS. 26A-26C is shown inverted from the position in use, when placed on a menstrual receptacle holder.

### Methods

In a second aspect, a method of storing a menstrual receptacle is provided. The method comprises placing a menstrual receptacle over the protrusion of any of the menstrual receptacle holders described in detail in the first aspect above.

Often, the menstrual receptacle is a menstrual cup or a menstrual disc.

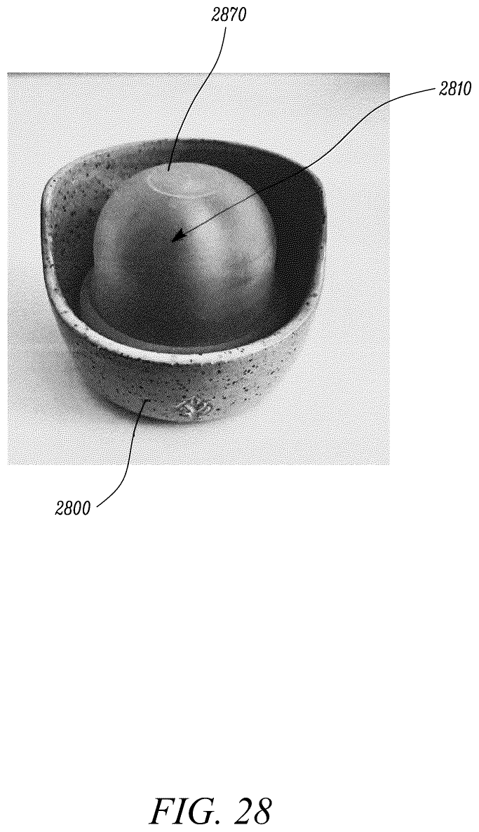

Referring to FIG. 27, a photograph is provided of an exemplary menstrual receptacle holder 2700 having a menstrual cup 2760 disposed on a protrusion 2710. This holder 2700 is the same as the menstrual receptacle holder 2500f shown in FIG. 25F. Referring to FIG. 28, a photograph is provided of an exemplary menstrual receptacle holder 2800 having a menstrual cup 2870 disposed on a protrusion 2810. This holder 2800 is the same as the menstrual receptacle holder 2500f shown in FIG. 25F.

In some embodiments, the protrusion is longer than a length of an interior of the menstrual receptacle, which helps prevent the menstrual receptacle from coming into contact with a surface on which the menstrual receptacle holder is placed or even into contact with a base of the menstrual receptacle holder.

An advantage of using a menstrual receptacle holder according to embodiments of the present disclosure is that it will not absorb fluids if a wet menstrual receptacle is placed on the holder (e.g., still wet from being cleaned or having fluid residue from being used). Accordingly, the menstrual receptacle holder will not promote the growth of bacteria that may be associated with having absorbed fluids.

An advantage of using a menstrual receptacle holder according to at least certain embodiments of the present disclosure is that after performing a cleaning of a menstrual receptacle in boiling water, the still hot menstrual receptacle may be placed on the protrusion of the holder without being damaged. For instance, shortly after being removed from boiling water, a menstrual receptacle may exhibit a temperature that is still greater than 150 degrees Fahrenheit, which could be damaging to temperature-sensitive materials if the menstrual receptacle were to be placed thereon.

In a third aspect, a method of cleaning a menstrual receptacle is provided. The method comprises applying a cleaner to at least one of the protrusion of the menstrual receptacle holder of any of the menstrual receptacle holders described in detail in the first aspect above or a menstrual receptacle; and placing the menstrual receptacle on the protrusion. As such, in some embodiments a cleaner is applied to just the protrusion of the menstrual receptacle holder, in some embodiments a cleaner is applied to just a menstrual receptacle (e.g., an interior surface of the menstrual receptacle), and in some embodiments a cleaner is applied to both the protrusion of the menstrual receptacle holder and the menstrual receptacle. In some embodiments, selecting a menstrual receptacle holder that has a wall may be helpful in effectively cleaning the menstrual receptacle.

Optionally, the method further includes turning the menstrual receptacle in at least one direction while the menstrual receptacle is located over the protrusion. In select embodiments, it may be particularly useful for the protrusion to have a plurality of horizontal utilitarian protrusions and indentations, such as those provided on the menstrual receptacle holder depicted in FIGS. 3 and 20, to assist in cleaning material out of an interior of the menstrual receptacle.

In some embodiments, the method further includes rinsing the menstrual receptacle.

In some cases, the method further includes leaving the menstrual receptacle on the menstrual receptacle holder for drying.

When performing methods that involve cleaning a menstrual receptacle and/or placing a clean wet menstrual receptacle on a holder, it may be advantageous to use a menstrual receptacle holder that includes at least one void present through a thickness of a portion of the menstrual receptacle holder to facilitate drainage of cleaning solution, water, etc., such as exemplified in each of FIGS. 14-16, 21, and 23.

### Listing of Exemplary Embodiments

In a first embodiment, the present disclosure provides a menstrual receptacle holder comprising a structure comprising a protrusion that is configured to support a menstrual receptacle, wherein the protrusion is formed of a nonporous material.

In a second embodiment, the present disclosure provides a menstrual receptacle holder according to the first embodiment, wherein the nonporous material comprises a porous material having an impermeable coating on a major surface thereof.

In a third embodiment, the present disclosure provides a menstrual receptacle holder according to the second embodiment, wherein the nonporous material comprises a glazed ceramic.

In a fourth embodiment, the present disclosure provides a menstrual receptacle holder according to any of the first through third embodiments, wherein the protrusion has a curvilinear cross-sectional shape.

In a fifth embodiment, the present disclosure provides a menstrual receptacle holder according to any of the first through fourth embodiments, wherein the protrusion defines a hollow body defining an open first end and an open opposing second end.

In a sixth embodiment, the present disclosure provides a menstrual receptacle holder according to any of the first through fifth embodiments, wherein the protrusion has a largest outer diameter of between 10 millimeters (mm) to 70 mm.

In a seventh embodiment, the present disclosure provides a menstrual receptacle holder according to any of the first through sixth embodiments, wherein the protrusion has a main body that is free of utilitarian projections and indentations.

In an eighth embodiment, the present disclosure provides a menstrual receptacle holder according to any of the first through seventh embodiments, wherein the protrusion has a shape that is conical, frustoconical, or cylindrical.

In a ninth embodiment, the present disclosure provides a menstrual receptacle holder according to any of the first through eighth embodiments, wherein the protrusion has a length of 20 mm to 100 mm.

In a tenth embodiment, the present disclosure provides a menstrual receptacle holder according to any of the first through ninth embodiments, wherein the structure consists of the protrusion.

In an eleventh embodiment, the present disclosure provides a menstrual receptacle holder according to the tenth embodiment, wherein the protrusion has a length of 40 mm to 100 mm.

In a twelfth embodiment, the present disclosure provides a menstrual receptacle holder according to any of the first through ninth embodiments, wherein the protrusion comprises a first end and an opposing second end and wherein the structure further comprises a base attached to the second end of the protrusion.

In a thirteenth embodiment, the present disclosure provides a menstrual receptacle holder according to the twelfth embodiment, wherein the base comprises a portion that extends in a direction away from a center point of the protrusion.

In a fourteenth embodiment, the present disclosure provides a menstrual receptacle holder according to the thirteenth embodiment, wherein the base further comprises a wall that extends in a direction away from the portion and provides an exterior structure of the menstrual receptacle holder.

In a fifteenth embodiment, the present disclosure provides a menstrual receptacle holder according to the fourteenth embodiment, wherein the wall has a single height.

In a sixteenth embodiment, the present disclosure provides a menstrual receptacle holder according to the fourteenth embodiment, wherein the wall has two or more different heights.

In a seventeenth embodiment, the present disclosure provides a menstrual receptacle holder according to any of the fourteenth through sixteenth embodiments, wherein the wall defines at least one void through a thickness of the wall.

In an eighteenth embodiment, the present disclosure provides a menstrual receptacle holder according to any of the thirteenth through seventeenth embodiments, wherein the portion of the base that extends in a direction away from a center point of the protrusion defines at least one void through a thickness of the portion.

In a nineteenth embodiment, the present disclosure provides a menstrual receptacle holder according to any of the thirteenth through eighteenth embodiments, wherein the protrusion defines at least one void through a thickness of the protrusion.

In a twentieth embodiment, the present disclosure provides a menstrual receptacle holder according to any of the thirteenth through nineteenth embodiments, further comprising a tray disposed below the base.

In a twenty-first embodiment, the present disclosure provides a menstrual receptacle holder according to the twentieth embodiment, wherein the tray is attached to the base.

In a twenty-second embodiment, the present disclosure provides a menstrual receptacle holder according to any of the first through twenty-first embodiments, wherein an interior of the protrusion is configured to provide a finger grip for the menstrual receptacle holder.

In a twenty-third embodiment, the present disclosure provides a menstrual receptacle holder according to any of the first through twenty-first embodiments, wherein the protrusion is solid.

In a twenty-fourth embodiment, the present disclosure provides a menstrual receptacle holder according to any of the first through twenty-third embodiments, further comprising a cover configured to surround the protrusion.

In a twenty-fifth embodiment, the present disclosure provides a method of storing a menstrual receptacle, the method comprising placing a menstrual receptacle over the protrusion of any of the menstrual receptacle holders according to any of the first through twenty-fourth embodiments.

In a twenty-sixth embodiment, the present disclosure provides a method of storing a menstrual receptacle according to the twenty-fifth embodiment, wherein the menstrual receptacle is a menstrual cup or a menstrual disc.

In a twenty-seventh embodiment, the present disclosure provides a method of storing a menstrual receptacle according to the twenty-fifth or twenty-sixth embodiment, wherein the protrusion is longer than a length of an interior of the menstrual receptacle.

In a twenty-eighth embodiment, the present disclosure provides a method of storing a menstrual receptacle according to any of the twenty-fifth through twenty-seventh embodiments, wherein the menstrual receptacle has at least one of i) water on a surface of the menstrual receptacle or ii) exhibits a temperature of greater than 150 degrees Fahrenheit.

In a twenty-ninth embodiment, the present disclosure provides a method of cleaning a menstrual receptacle, comprising applying a cleaner to at least one of the protrusion of the menstrual receptacle holder according to any of the first through twenty-fourth embodiments or a menstrual receptacle; and placing the menstrual receptacle on the protrusion.

In a thirtieth embodiment, the present disclosure provides a method of cleaning a menstrual receptacle according to the twenty-ninth embodiment, further comprising turning the menstrual receptacle in at least one direction while the menstrual receptacle is located over the protrusion.

In a thirty-first embodiment, the present disclosure provides a method of cleaning a menstrual receptacle according to the twenty-ninth or thirtieth embodiment, further comprising rinsing the menstrual receptacle.

In a thirty-second embodiment, the present disclosure provides a method of cleaning a menstrual receptacle according to the thirty-first embodiment, further comprising leaving the menstrual receptacle on the menstrual receptacle holder for drying.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

What is claimed is:

1. A menstrual receptacle holder comprising:
a structure comprising a protrusion that is configured to support a menstrual receptacle, wherein the protrusion is formed of a nonporous material.

2. The menstrual receptacle holder of claim 1, wherein the nonporous material comprises a porous material having an impermeable coating on a major surface thereof.

3. The menstrual receptacle holder of claim 2, wherein the nonporous material comprises a glazed ceramic.

4. The menstrual receptacle holder of claim 1, wherein the protrusion has a curvilinear cross-sectional shape.

5. The menstrual receptacle holder of claim 1, wherein the protrusion defines a hollow body defining an open first end and an open opposing second end.

6. The menstrual receptacle holder of claim 1, wherein the protrusion has a largest outer diameter of between 10 millimeters (mm) to 70 mm.

7. The menstrual receptacle holder of claim 1, wherein the protrusion has a main body that is free of utilitarian projections and indentations.

8. The menstrual receptacle holder of claim 1, wherein the protrusion has a shape that is conical, frustoconical, or cylindrical.

9. The menstrual receptacle holder of claim 1, wherein the protrusion has a length of 20 mm to 100 mm.

10. The menstrual receptacle holder of claim 1, wherein the structure consists of the protrusion.

11. The menstrual receptacle holder of claim 1, wherein the protrusion comprises a first end and an opposing second end and wherein the structure further comprises a base attached to the second end of the protrusion.

12. The menstrual receptacle holder of claim 11, wherein the base comprises a portion that extends in a direction away from a center point of the protrusion.

13. The menstrual receptacle holder of claim 12, wherein the base further comprises a wall that extends in a direction away from the portion and provides an exterior structure of the menstrual receptacle holder.

14. The menstrual receptacle holder of claim 13, wherein the wall has two or more different heights.

15. The menstrual receptacle holder of claim 13, further comprising at least one void, wherein the wall defines at least one void through a thickness of the wall, wherein the portion of the base that extends in a direction away from a center point of the protrusion defines at least one void through a thickness of the portion, and/or wherein the protrusion defines at least one void through a thickness of the protrusion.

16. The menstrual receptacle holder of claim 1, further comprising a tray disposed below the base, wherein the tray is attached to the base.

17. The menstrual receptacle holder of claim 1, wherein an interior of the protrusion is configured to provide a finger grip for the menstrual receptacle holder.

18. The menstrual receptacle holder of claim 1, wherein the protrusion is solid.

19. A method of storing a menstrual receptacle, the method comprising placing a menstrual receptacle over the protrusion of the menstrual receptacle holder of claim 1.

20. A method of cleaning a menstrual receptacle, the method comprising:
applying a cleaner to at least one of the protrusion of the menstrual receptacle holder of claim 1 or a menstrual receptacle; and
placing the menstrual receptacle on the protrusion.
